(19) United States
(12) Patent Application Publication
(10) Pub. No.: US 2025/0134425 A1
(43) Pub. Date: May 1, 2025

(54) ACTIVE-PULSE BLOOD ANALYSIS SYSTEM

(71) Applicant: Masimo Corporation, Irvine, CA (US)

(72) Inventors: Massi Joe E. Kiani, Laguna Niguel, CA (US); Mathew Paul, Irvine, CA (US); Jesse Chen, Foothill Ranch, CA (US); Marcelo M. Lamego, Cupertino, CA (US)

(21) Appl. No.: 18/987,361

(22) Filed: Dec. 19, 2024

Related U.S. Application Data
(63) Continuation of application No. 18/496,255, filed on Oct. 27, 2023, now Pat. No. 12,193,813.
Application No. 18/496,255 is a continuation of application No. 17/576,281, filed on Jan. 14, 2022, now Pat. No. 11,839,470.
Application No. 17/576,281 is a continuation of application No. 16/804,507, filed on Feb. 28, 2020, now Pat. No. 11,224,363.
Application No. 16/804,507 is a continuation of application No. 15/670,405, filed on Aug. 7, 2017, now Pat. No. 10,610,139.
Application No. 15/670,405 is a continuation of application No. 14/328,694, filed on Jul. 10, 2014, now Pat. No. 9,724,025.
Application No. 14/328,694 is a continuation-in-part of application No. 14/153,393, filed on Jan. 13, 2014, now abandoned.
(60) Provisional application No. 61/752,976, filed on Jan. 16, 2013.
(60) Provisional application No. 61/844,699, filed on Jul. 10, 2013.

(51) Int. Cl. A61B 5/1455; A61B 5/00

(52) U.S. Cl. CPC A61B 5/14552; A61B 5/6826; A61B 5/7207; A61B 5/721

(57) ABSTRACT

An active-pulse blood analysis system has an optical sensor that illuminates a tissue site with multiple wavelengths of optical radiation and outputs sensor signals responsive to the optical radiation after attenuation by pulsatile blood flow within the tissue site. A monitor communicates with the sensor signals and is responsive to arterial pulses within a first bandwidth and active pulses within a second bandwidth so as to generate arterial pulse ratios and active pulse ratios according to the wavelengths. An arterial calibration curve relates the arterial pulse ratios to a first arterial oxygen saturation value and an active pulse calibration curve relates the active pulse ratios to a second arterial oxygen saturation value. Decision logic outputs one of the first and second arterial oxygen saturation values based upon perfusion and signal quality.

### PRIORITY CLAIM AND REFERENCE TO RELATED APPLICATIONS

The present application is a continuation-in-part of U.S. patent application Ser. No. 14/153,393, filed Jan. 13, 2014, titled Active-Pulse Blood Analysis System, which claims priority benefit under 35 U.S.C. § 119(e) to U.S. Provisional Patent Application Ser. No. 61/752,976, filed Jan. 16, 2013, titled Active-Pulse Blood Analysis System; the present application claims priority benefit under 35 U.S.C. § 119(e) to U.S. Provisional Patent Application Ser. No. 61/844,699, filed Jul. 10, 2013, titled Active-Pulse Blood Analysis System; the above-referenced patent application and provisional patent applications are hereby incorporated in their entireties by reference herein.

### BACKGROUND OF THE INVENTION

Noninvasive physiological monitoring systems for measuring constituents of circulating blood have advanced from basic pulse oximeters to monitors capable of measuring abnormal and total hemoglobin among other parameters. A basic pulse oximeter capable of measuring blood oxygen saturation typically includes an optical sensor, a monitor for processing sensor signals and displaying results and a cable electrically interconnecting the sensor and the monitor. A pulse oximetry sensor typically has a red wavelength light emitting diode (LED), an infrared (IR) wavelength LED and a photodiode detector. The LEDs and detector are attached to a patient tissue site, such as a finger. The cable transmits drive signals from the monitor to the LEDs, and the LEDs respond to the drive signals to transmit light into the tissue site. The detector generates a photoplethysmograph signal responsive to the emitted light after attenuation by pulsatile blood flow within the tissue site. The cable transmits the detector signal to the monitor, which processes the signal to provide a numerical readout of oxygen saturation (SpO₂) and pulse rate, along with an audible pulse indication of the person's pulse. The photoplethysmograph waveform may also be displayed.

### SUMMARY OF THE INVENTION

Conventional pulse oximetry assumes that arterial blood is the only pulsatile blood flow in the measurement site. During patient motion, venous blood also moves, which causes errors in conventional pulse oximetry. Advanced pulse oximetry processes the venous blood signal so as to report true arterial oxygen saturation and pulse rate under conditions of patient movement. Advanced pulse oximetry also functions under conditions of low perfusion (small signal amplitude), intense ambient light (artificial or sunlight) and electrosurgical instrument interference, which are scenarios where conventional pulse oximetry tends to fail.

Advanced pulse oximetry is described in at least U.S. Pat. Nos. 6,770,028; 6,658,276; 6,157,850; 6,002,952; 5,769,785 and 5,758,644, which are assigned to Masimo Corporation (“Masimo”) of Irvine, California and are incorporated in their entireties by reference herein. Corresponding low noise optical sensors are disclosed in at least U.S. Pat. Nos. 6,985,764; 6,813,511; 6,792,300; 6,256,523; 6,088,607; 5,782,757 and 5,638,818, which are also assigned to Masimo and are also incorporated in their entireties by reference herein. Advanced pulse oximetry systems including Masimo SET® low noise optical sensors and read through motion pulse oximetry monitors for measuring SpO₂, pulse rate (PR) and perfusion index (PI) are available from Masimo. Optical sensors include any of Masimo LNOP®, LNCS®, SofTouch™ and Blue™ adhesive or reusable sensors. Pulse oximetry monitors include any of Masimo Rad-8®, Rad-5®, Rad®-5v or SatShare® monitors.

Advanced blood parameter measurement systems are described in at least U.S. Pat. 7,647,083, filed Mar. 1, 2006, titled Multiple Wavelength Sensor Equalization; U.S. Pat. No. 7,729,733, filed Mar. 1, 2006, titled Configurable Physiological Measurement System; U.S. Pat. Pub. No. 2006/0211925, filed Mar. 1, 2006, titled Physiological Parameter Confidence Measure and U.S. Pat. Pub. No. 2006/0238358, filed Mar. 1, 2006, titled Noninvasive Multi-Parameter Patient Monitor, all assigned to Cercacor Laboratories, Inc., Irvine, CA (“Cercacor”) and all incorporated in their entireties by reference herein. An advanced parameter measurement system that includes acoustic monitoring is described in U.S. Pat. Pub. No. 2010/0274099, filed Dec. 21, 2009, titled Acoustic Sensor Assembly, assigned to Masimo and incorporated in its entirety by reference herein.

Advanced blood parameter measurement systems include Masimo Rainbow® SET, which provides measurements in addition to SpO₂, such as total hemoglobin (SpHb™), oxygen content (SpOC™), methemoglobin (SpMet®), carboxyhemoglobin (SpCO®) and PVI®. Advanced blood parameter sensors include Masimo Rainbow® adhesive, ReSposable™ and reusable sensors. Advanced blood parameter monitors include Masimo Radical-7™, Rad-87™ and Rad-57™ monitors, all available from Masimo. Advanced parameter measurement systems may also include acoustic monitoring such as acoustic respiration rate (RRa™) using a Rainbow Acoustic Sensor™ and Rad-87™ monitor, available from Masimo. Such advanced pulse oximeters, low noise sensors and advanced parameter systems have gained rapid acceptance in a wide variety of medical applications, including surgical wards, intensive care and neonatal units, general wards, home care, physical training, and virtually all types of monitoring scenarios.

One aspect of an active-pulse blood analysis system has an optical sensor that illuminates a tissue site with multiple wavelengths of optical radiation and that outputs sensor signals responsive to the optical radiation after attenuation by pulsatile blood flow within the tissue site. A monitor communicates with the sensor signals and is responsive to arterial pulses within a first bandwidth and active pulses within a second bandwidth so as to generate arterial pulse ratios and active pulse ratios according to the wavelengths. An arterial calibration curve relates the arterial pulse ratios to a first arterial oxygen saturation, and a first active pulse calibration curve relates the active pulse ratios to a first venous oxygen saturation.

In various embodiments, the arterial calibration curve relates the active pulse ratios to a second venous oxygen saturation. A second active pulse calibration curve relates the active pulse ratios to a second arterial oxygen saturation. A multiplexer selects from the first arterial oxygen saturation and the second arterial oxygen saturation so as to output a third arterial oxygen saturation. A decision logic determines the third arterial oxygen saturation. The decision logic receives a motion input and a perfusion input. The decision logic selects the third arterial oxygen saturation when perfusion is in a lower range of perfusion values and motion is in a higher range of motion values.

Another aspect of an active-pulse blood analysis system inputs optical sensor data, filters the sensor data into arterial pulse data at a lower range of frequencies and active pulse data at a higher range of frequencies, calculates arterial pulse ratios from the arterial pulse data and active pulse ratios from the active pulse data, applies an arterial calibration curve to the arterial pulse ratios so as to generate an SpO₂ parameter and applies a second calibration curve so as to generate a second oxygen saturation parameter. In various embodiments, the second calibration curve is a venous calibration curve and the second oxygen saturation parameter is SpvO₂, the second calibration curve is an arterial calibration curve and the second oxygen saturation parameter is SpvO₂^{A}, the second calibration curve relates active pulse ratio data to SaO₂ values so as to define an arterial saturation parameter SpO₂^{AP}.

In various other embodiments, one of the SpO₂ parameter and the SpO₂^{AP} are output according to a motion and perfusion selection criterion. The selection criterion is based upon motion zones and perfusion zones. The selection criterion is based upon a boundary between a first area of relatively high perfusion combined with relatively little motion and a second area of relatively low perfusion combined with relatively large motion.

A further aspect of an active-pulse blood analysis system is an optical sensor for transmitting multiple wavelengths of light into a tissue site and detecting the transmitted light after attenuation by arterial blood flow and active pulse blood flow within the tissue site so as to generate plethysmograph data. A filter separates the detected plethysmograph data into arterial pulse data and active pulse data. A processor calculates arterial ratios from the arterial pulse data and active pulse ratios from the active pulse data. An arterial calibration curve relates the arterial pulse ratios to SpO₂ values, and a venous calibration curve relates the active pulse ratios to SpvO₂ values. In various embodiments, an arterial cal curve relates the active pulse ratios to SpvO₂^{A} values, an active pulse cal curve relates the active pulse ratios to SpO₂^{AP} values, a multiplexor relates SpO₂ and SpO₂^{AP} values to SpO₂^{M} values, a decision logic selects SpO₂ and SpO₂^{AP} to output as SpO₂^{M} according to a combination of motion and perfusion, and a zone specifies the decision logic according to motion and perfusion.

Yet another aspect of an active-pulse blood analysis system is an optical sensor that illuminates a tissue site with multiple wavelengths of optical radiation and that outputs sensor signals responsive to the optical radiation after attenuation by pulsatile blood flow within the tissue site. A monitor communicates with the sensor signals and is responsive to arterial pulses within a first bandwidth and active pulses within a second bandwidth so as to generate arterial pulse ratios and active pulse ratios according to the wavelengths. An arterial calibration curve relates the arterial pulse ratios to a first arterial oxygen saturation (SpO₂), and an active pulse calibration curve relates the active pulse ratios to a second arterial oxygen saturation (SpO₂^{AP}).

In various embodiments, a multiplexer has a third arterial oxygen saturation (SpO₂^{M}) output selected from one of the first arterial oxygen saturation and the second arterial oxygen saturation. A decision logic determines the third arterial oxygen saturation. Signal quality and perfusion are input to the decision logic. The decision logic selects the second arterial oxygen saturation when perfusion is in a lower range of perfusion values and signal quality is in a lower range of signal quality values. The decision logic inputs a Boolean perfusion value (BPI) and a Boolean signal quality value (BSQ).

An additional aspect of an active-pulse blood analysis system is inputting optical sensor data, filtering the optical sensor data into arterial pulse data at a lower range of frequencies and active pulse data at a higher range of frequencies, calculating arterial pulse ratios from the arterial pulse data. Active pulse ratios are calculated from the active pulse data. An arterial calibration curve is applied to the arterial pulse ratios so as to generate an SpO₂ parameter indicative of arterial oxygen saturation determined from an arterial pulse. An active pulse calibration curve is applied to the active pulse ratios so as to generate an SpO₂^{AP} parameter indicative of arterial oxygen saturation determined from an active pulse.

In various embodiments, active-pulse blood analysis comprises multiplexing the SpO₂ parameter and the SpO₂^{AP} parameter so as to generate an SpO₂ output parameter indicative of an arterial oxygen saturation measurement tolerate to at least one of motion, low perfusion and low signal quality. Multiplexing comprises selecting one of the SpO₂ parameter and the SpO₂^{AP} parameter as the SpO₂^{M} output parameter according to a combination of a signal quality input and a perfusion index input. Selecting comprises outputting SpO₂^{AP} as the SpO₂^{M} output parameter when the combination of signal quality and perfusion are below a threshold boundary. Selecting comprises outputting SpO₂ as the SpO₂^{M} output parameter when the combination of signal quality and perfusion are above the threshold boundary. The threshold boundary is specified by discrete zones of signal quality and perfusion. The threshold boundary is specified by a continuous curve that is a function of signal quality and perfusion.

Further aspects of an active-pulse blood analysis apparatus comprise an optical sensor means for transmitting multiple wavelengths of light into a tissue site and detecting the transmitted light after attenuation by arterial blood flow and active pulsed blood flow within the tissue site so as to generate plethysmograph data. A filter means separates the detected plethysmograph data into arterial pulse data and active pulse data. A processor means calculates arterial ratios from the arterial pulse data and active pulse ratios from the active pulse data. An arterial calibration curve means relates the arterial pulse ratios to oxygen saturation values (SpO₂). An active pulse calibration curve means relates the active pulse ratios to active pulse oxygen saturation values (SpO₂^{AP}).

In various embodiments, the active-pulse blood analysis apparatus a further comprising a multiplexer means for combining the oxygen saturation values and active pulse oxygen saturation values into multiplexed oxygen saturation values (SpO₂^{M}). A decision logic means selects from SpO₂ and SpO₂^{AP} as the SpO₂^{M} output. The decision logic means is responsive to at least two of motion, perfusion and signal quality inputs.

### BRIEF DESCRIPTION OF THE DRAWINGS

FIG. 1 is an illustration of an active-pulse blood analysis system for concurrently determining a person's arterial oxygen saturation (SpO₂) and venous oxygen saturation (SpvO₂);

FIGS. 2A-8 are illustrations of active-pulse blood analysis techniques;

FIG. 2A illustrates a prior art occlusive, off-site active-pulse technique for temporally-spaced (non-concurrent) arterial and venous oxygen saturation measurements;

FIG. 28 illustrates a non-occlusive, on-site active-pulse technique for concurrent SpO₂ and SpvO₂ measurements;

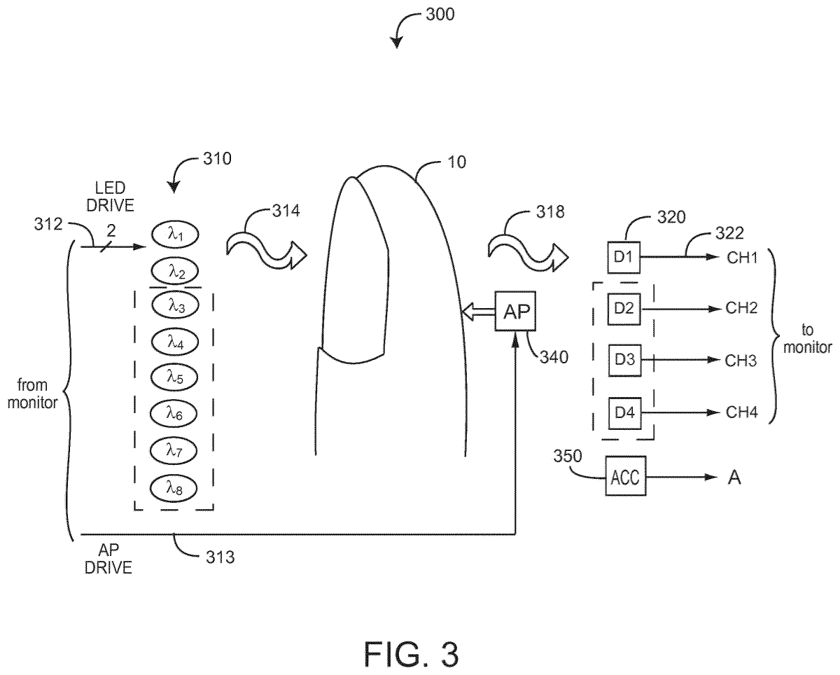

FIG. 3 is an illustration of an active-pulse blood analysis sensor that allows concurrent arterial-pulse and active-pulse blood analysis;

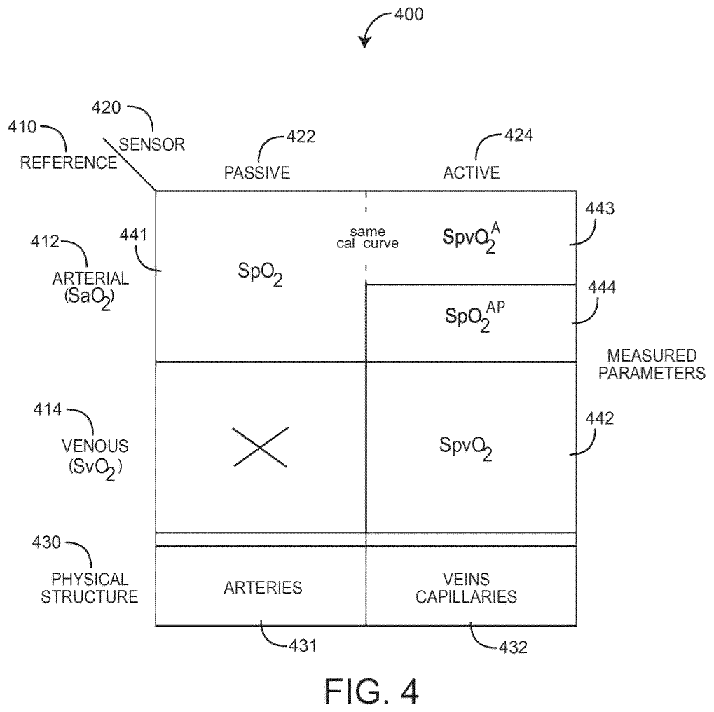

FIG. 4 is a relational chart for various active-pulse blood analysis parameters;

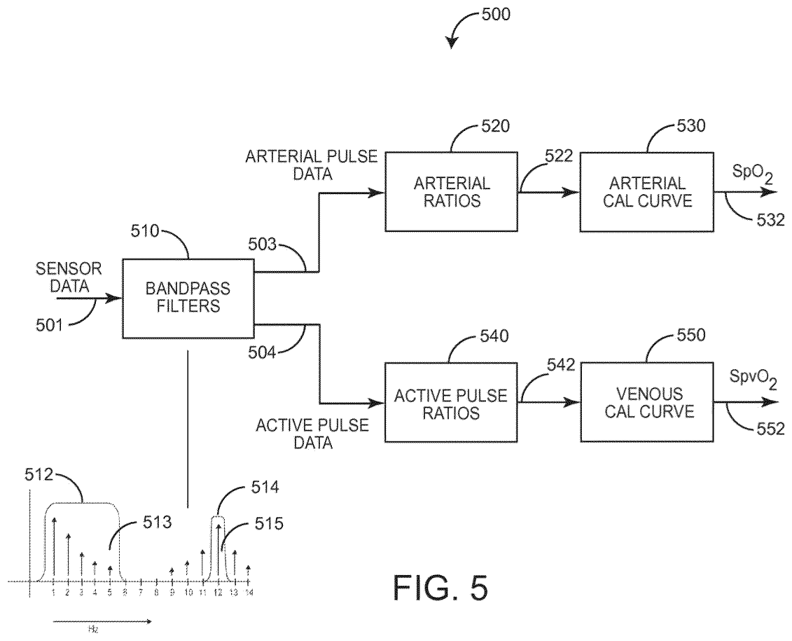

FIG. 5 is a block diagram of active-pulse blood analysis for determining SpO₂ using an arterial cal curve and SpvO₂ using a venous cal curve;

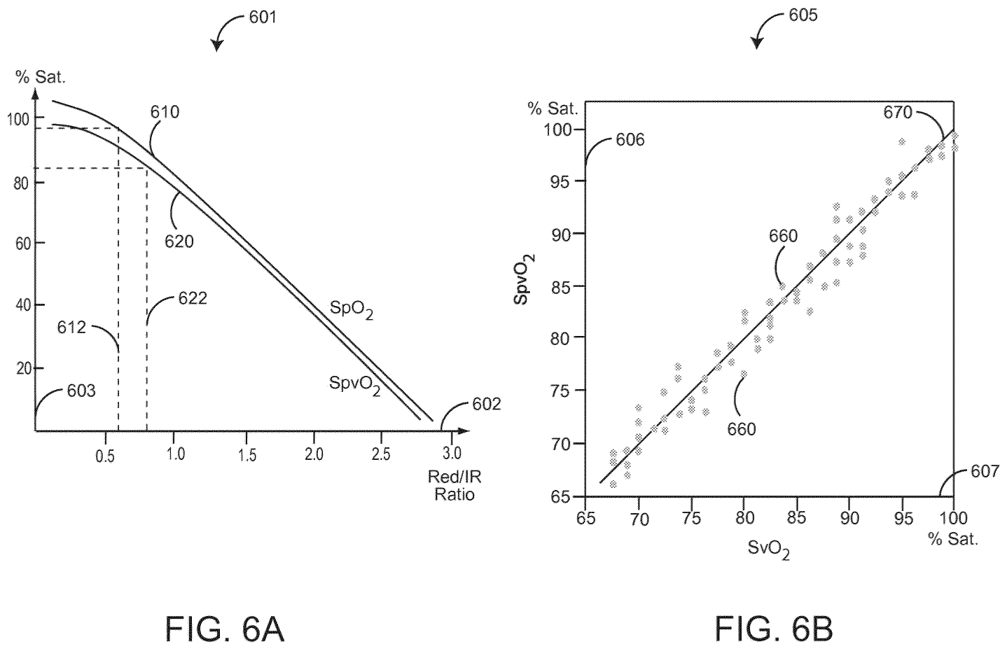

FIGS. 6A-B are graphs of active-pulse blood analysis calibration curves (cal curves);

FIG. 6A is a graph of two-dimensional SpO₂ and SpvO₂ cal curves;

FIG. 6B is a graph of a multi-dimensional SpvO₂ cal curve;

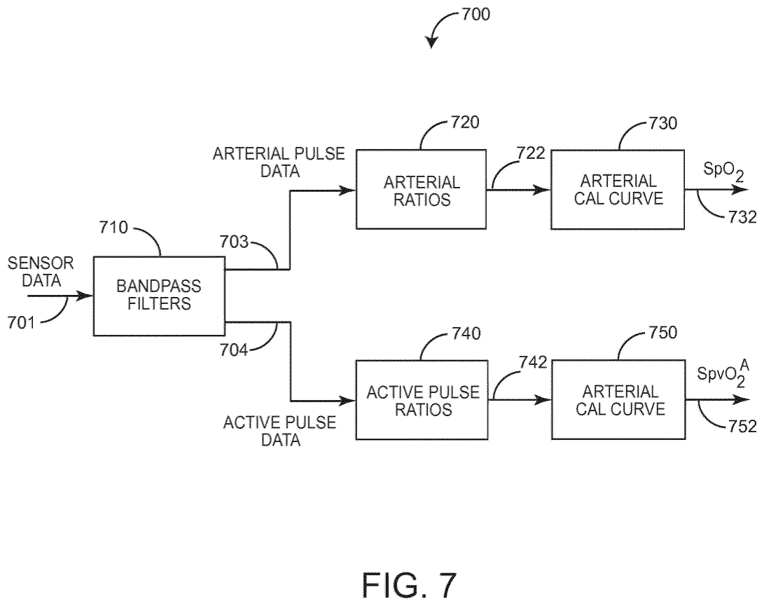

FIG. 7 is a block diagram of active-pulse blood analysis for determining SpO₂ and SpvO₂^{A} using the same arterial calibration curve;

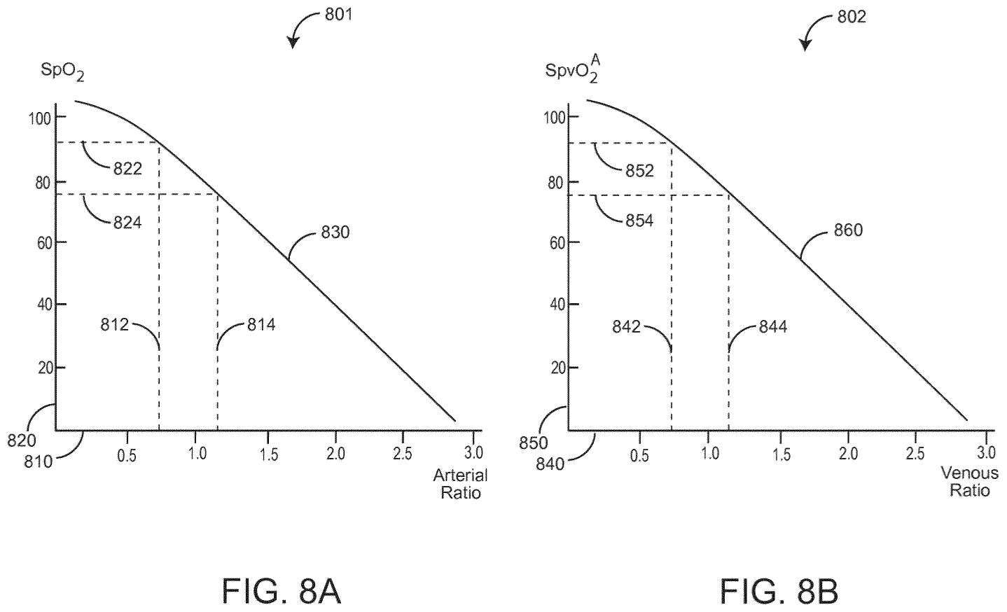

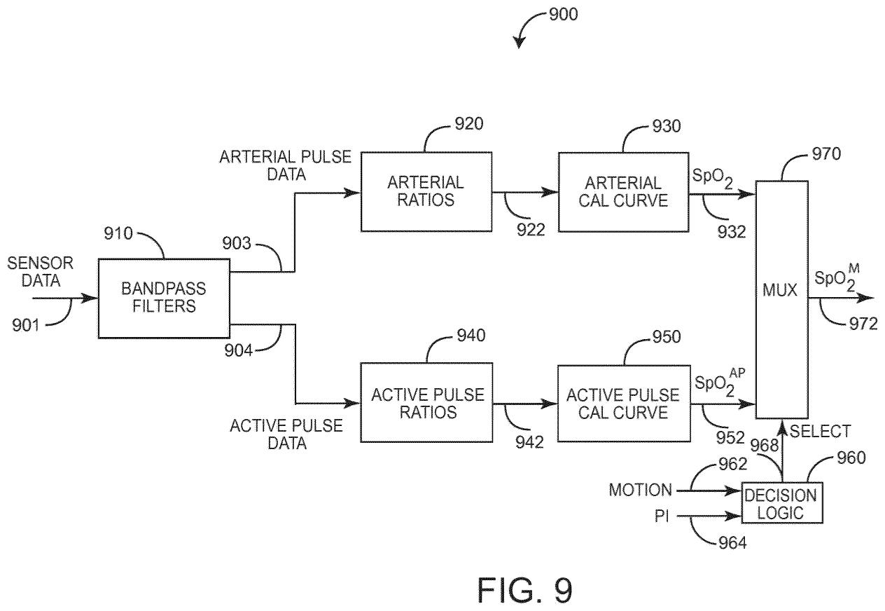

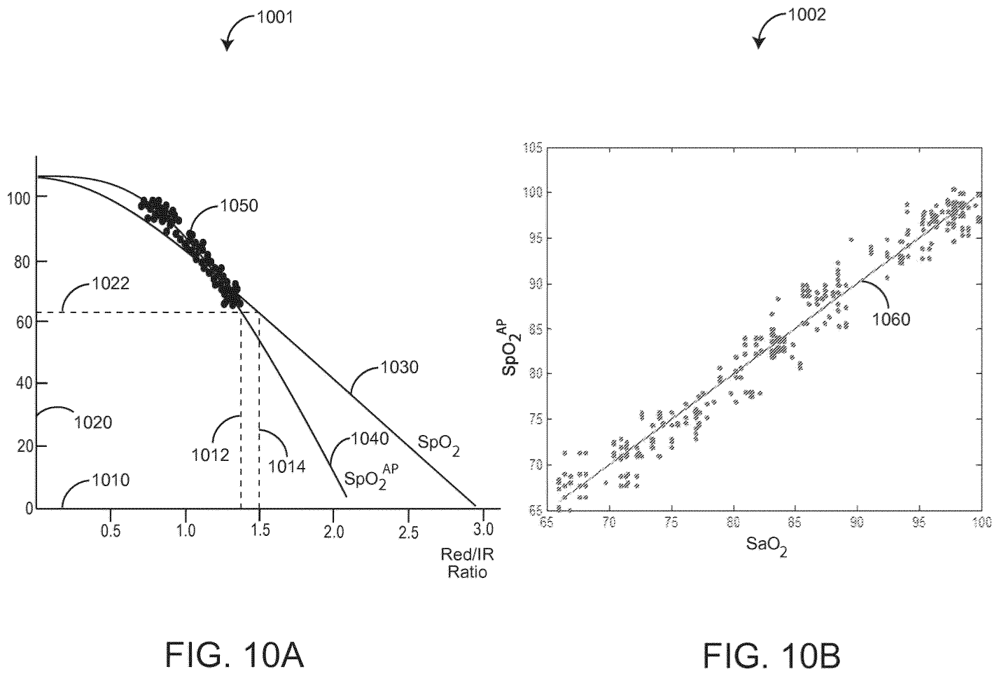

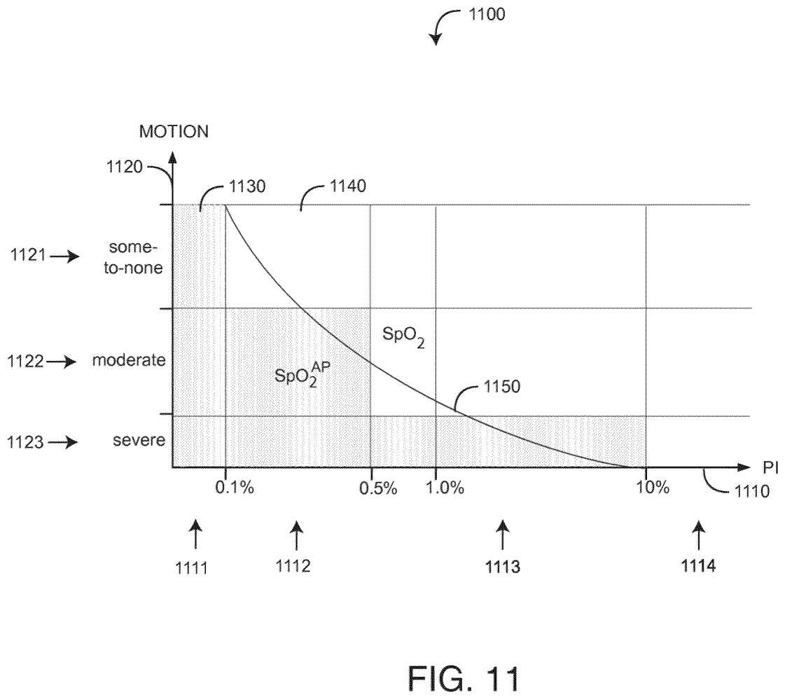

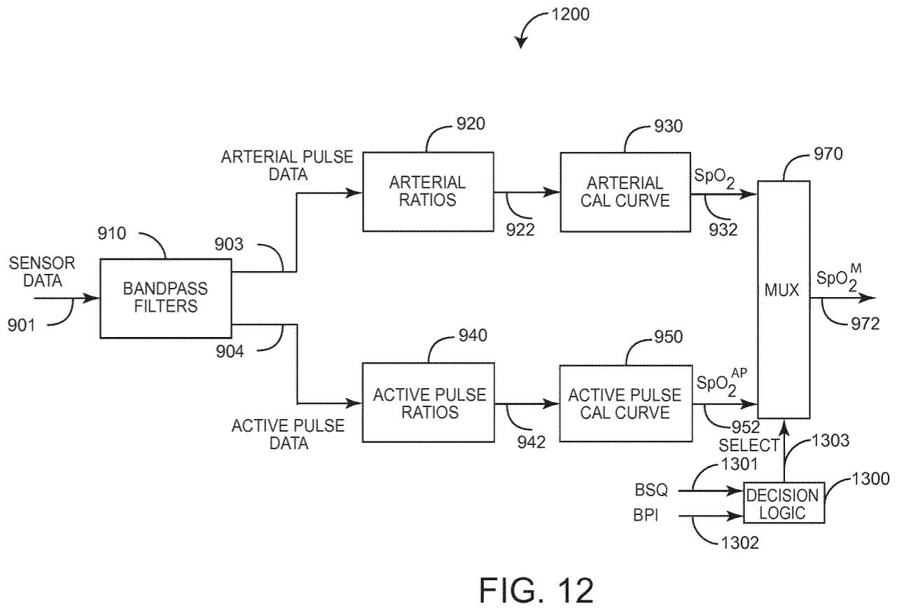

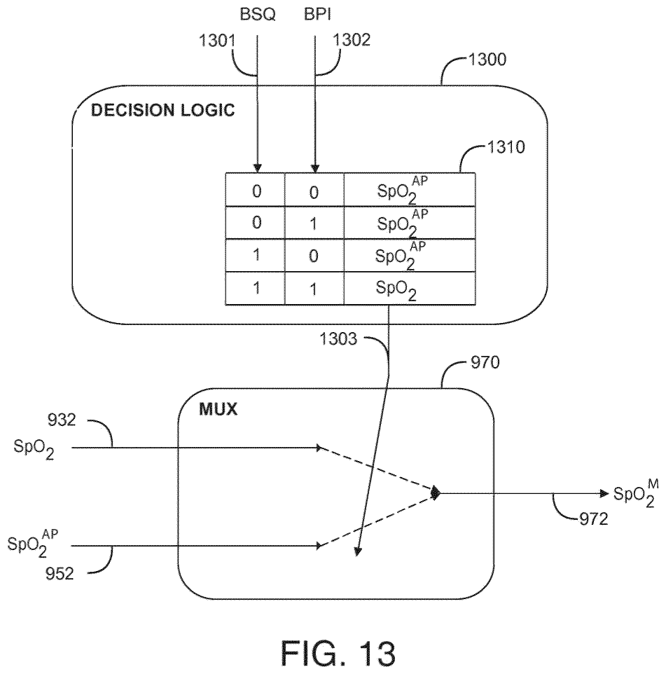

FIGS. 8A-B are graphs of active-pulse blood analysis cal curves for calculating both SpO₂ and SpvO₂^{A};

FIG. 8A is a graph of an arterial cal curve for calculating SpO₂; and

FIG. 8B is a graph of an identical arterial cal curve for calculating SpvO₂^{A};

FIG. 9 is a block diagram of active-pulse blood analysis for determining SpO₂ and SpO₂^{AP} and for combining SpO₂ and SpO₂^{AP} based upon motion and perfusion index (PI) parameters so as to calculate a motion and low perfusion tolerant measure of arterial oxygen saturation (SpO₂^{M});

FIGS. 10A-B are graphs of active-pulse blood analysis cal curves for calculating SpO₂ and SpO₂^{AP};

FIG. 10A is a two-dimensional SpO₂^{AP} cal curve shown in relation to a SpO₂ cal curve; and

FIG. 10B is a multidimensional SpO₂^{AP} cal curve;

FIG. 11 is a motion versus perfusion decision graph for combining SpO₂and SpO₂^{AP} so as to calculate a motion and low perfusion tolerant measure of arterial oxygen saturation (SpO₂^{M});

FIG. 12 is a block diagram of active-pulse blood analysis for determining SpO₂ and SpO₂^{AP} and for combining SpO₂ and SpO₂^{AP} based upon BSQ (Boolean signal quality) and BPI (Boolean perfusion index) parameters so as to calculate a motion and low perfusion tolerant measure of arterial oxygen saturation (SpO₂^{M}); and

FIG. 13 is a block diagram of a decision logic embodiment for combining SpO₂ and SpO₂^{AP} based upon BSQ and BPI so as to calculate SpO₂^{M}.

### DETAILED DESCRIPTION OF THE PREFERRED EMBODIMENTS

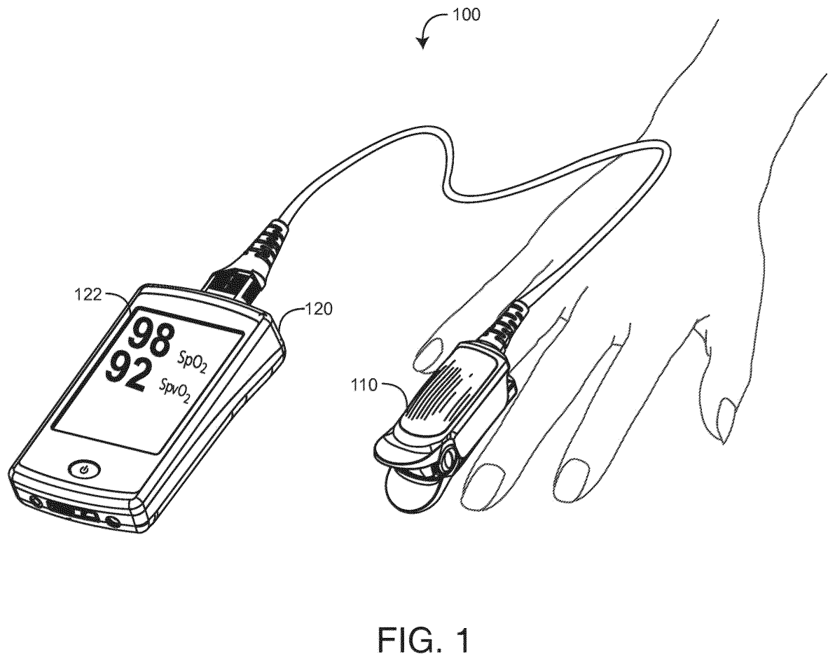

FIG. 1 illustrates an active-pulse blood analysis system 100 for concurrently determining a person's arterial oxygen saturation (SpO₂) and venous oxygen saturation (SpvO₂). The active-pulse blood analysis system 100 has an optical sensor 110 that transmits optical radiation at two or more wavelengths including red and infrared wavelengths. The active-pulse blood analysis system 100 also has a monitor 120 that determines the relative concentrations of blood constituents flowing in optically-probed pulsatile arteries and actively-pulsed capillaries and veins. A monitor display 122 is configured to readout concurrently measured oxygen saturation values including SpO₂, SpvO₂, SpvO₂^{A}, SpO₂^{AP} and SpO₂^{M}, as described below. A non-invasive blood analysis system utilizing an optical, active-pulse sensor is described in U.S. patent application Ser. No. 13/646,659 titled Noninvasive Blood Analysis System, filed Oct. 5, 2012, assigned to Cercacor and incorporated in its entirety by reference herein.

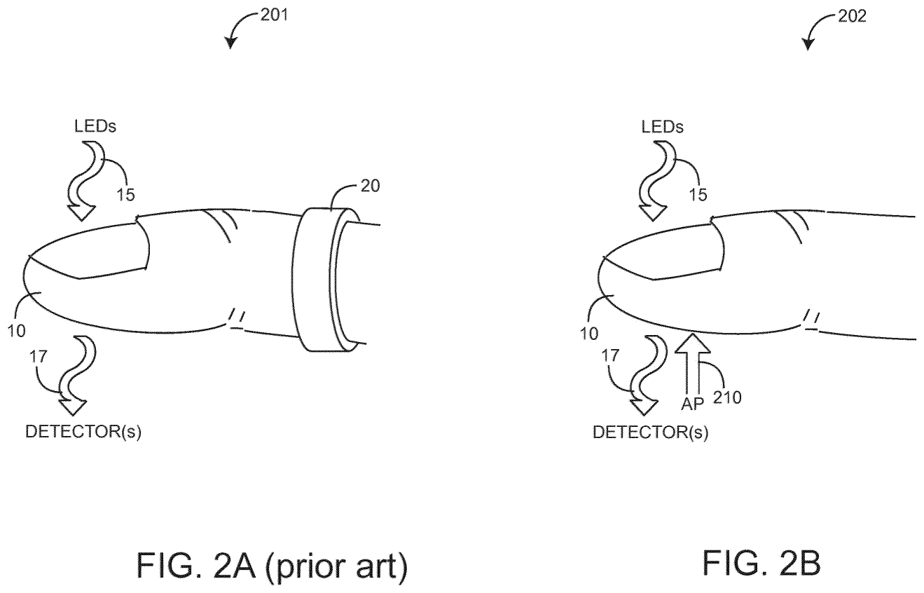

FIGS. 2A-B illustrate active-pulse blood analysis techniques. FIG. 2A illustrates a prior art occlusive, off-site active-pulse technique for temporally-spaced (non-concurrent) arterial and venous oxygen saturation measurements. A fingertip 10 is illuminated 15 with multiple wavelength light from, say, red and IR LEDs. Corresponding multiple wavelength light 17 emerges from the fingertip 10 after attenuation by pulsatile blood flow within the fingertip 10 and is received by detectors accordingly. The artificial pulse mechanism is a pressure cuff 20, as shown, or a plunger or similar mechanical device located distal the fingertip 10. An active-pulse sensor utilizing an off-site plunger or pressure cuff is described in U.S. Pat. No. 6,334,065, titled Stereo Pulse Oximeter, filed May 27, 1999, assigned to Masimo and incorporated in its entirety by reference herein. The downside to such an off-site active-pulse technique is that at least partial occlusion of the arterial blood flow occurs. As a result, accurate optical measurement of arterial blood constituents cannot be made concurrently with venous blood constituents. However, on-site active-pulse techniques present the difficulty of designing a mechanism that generates a pulse co-located with detectors, where the detected light tends to be sensitive to fingertip placement, vibration and movement. Further, conventional wisdom is that an on-site active (artificial) pulse alters or interferes with an arterial pulse such that concurrent measurement of arterial and venous blood constituents is infeasible.

FIG. 2B illustrates a non-occlusive, on-site active-pulse technique for concurrent SpO₂ and SpvO₂ measurements. In particular, a mechanical pulser 210 is co-located with sensor detectors at the fingertip 10 so that LED light 15 can be detected 17 after attenuation by pulsatile arterial, capillary and venous blood flow. An active-pulse optical sensor having mechanical, optical and electrical elements configured for concurrent probing of arterial, capillary and venous blood constituents is described in U.S. Patent Application No. 13/473,377, titled Personal Health Device, filed May 16, 2012, assigned to Cercacor and incorporated in its entirety by reference herein.

FIG. 3 illustrates an active-pulse blood analysis sensor 300 that allows concurrent natural pulse and active-pulse blood analysis. The sensor 300 has two or more LEDs (emitters) 310, one or more detectors 320 and an active-pulser 340. In other embodiments, the sensor 300 also has temperature sensors (not shown) responsive to the LEDs 310, the detector(s) 320 and the fingertip as well as an accelerometer 350 responsive to fingertip position and movement. The LEDs 310 are individually activated by LED drives 312 so as illuminate a tissue site 10 with optical radiation 314. The detector(s) 320 receive attenuated optical radiation 318 after absorption, reflection and diffusion by the tissue site 10 and by pulsatile blood flow within the tissue site 10. The active-pulse 340 has a motor that controls a mechanical pulser in response to an active-pulse drive signal 313. The motor has a “motor-on” state for starting the active-pulse and a “motor-off”' state for stopping the active-pulse. Accordingly, the pulsatile blood flow may be heart-pulsed arterial blood flow or actively-pulsed venous and capillary blood flow, or both. The detector(s) 320 generates one or more channels 322 of plethysmograph and active-pulse signals to a DSP (not shown) within the blood analysis monitor 120 (FIG. 1) for signal processing and analysis, as described in detail below.

FIG. 4 is a relational chart 400 for various active-pulse blood analysis parameters. The matrix rows 410 are invasive (blood draw) references. The matrix columns 420 are noninvasive sensor measurements. Each matrix cell 441-444 represents a blood parameter derived from an underlying calibration curve that correlates the invasive references 410 with the sensor measurements 420. FIGS. 6, 8 and 10, below, illustrate calibration curves corresponding to the cells 441-444. A “physical structure” row 430 appended at the bottom of the matrix 400 is a simple reminder that a passive sensor 422 “probes” the arteries 431, i.e. is responsive to heart-pulsed arterial blood flow, and that an active sensor 424 “probes” the capillaries and veins 432, i.e. is responsive to active-pulse induced venous blood flow. This calibration matrix 400 succinctly illustrates advantageously defined blood parameters listed within the cells 441-444, which are concurrently measured from a fingertip tissue site utilizing an active-pulse sensor 300 (FIG. 3).

As shown in FIG. 4, an SpaO₂ (or simply SpO₂) peripheral arterial oxygen saturation parameter 441 is a passive measurement 422 responsive to pulsatile arterial blood flow 431. An underlying SpO₂ calibration curve (“cal curve”) is generated from arterial blood draws 412 correlated with the sensor-derived measurements, as described with respect to FIG. 6A, below.

Also shown in FIG. 4, an SpvO₂ peripheral venous oxygen saturation parameter 442 is an active-pulse measurement 424, responsive to artificially-pulsed venous and capillary blood flow 432. An underlying SpvO₂ cal curve is generated from venous blood draws 414 correlated with the sensor-derived measurements, as described with respect to FIGS. 6A-B, below.

Further shown in FIG. 4, an SpvO₂^{A} peripheral venous oxygen saturation parameter 443 is an active-pulse measurement 424 responsive to artificially-pulsed venous and capillary blood flow 432. Advantageously, SpvO₂^{A} sensor measurements utilize the same arterial (“A”) cal curve 441 generated by passive sensor measurements 422 correlated with arterial blood draws 412, as cited above. SpvO₂^{A} measurements are described with respect to FIG. 8B, below.

Additionally shown in FIG. 4, an SpO₂^{AP} peripheral arterial oxygen saturation parameter 444 is an active-pulse measurement 424 responsive to artificially-pulsed venous and capillary blood flow 432 measured with an active-pulse sensor. Advantageously, SpO₂^{AP} sensor measurements 444 utilize a unique active-pulse (“AP”) cal curve generated from arterial blood draws 412 correlated with active-pulse sensor measurements, as described with respect to FIGS. 10A-B, below.

FIG. 5 illustrates an active-pulse blood analysis system 500 embodiment having a sensor data input 501, an SpO₂ 532 output and an SpvO₂ 552 output. The sensor data 501 input has arterial pulse components 513 and active-pulse components 515. Resting heart rates range around 60 bpm (1 Hz). As such, a typical arterial pulse includes a fundamental around 1 Hz and harmonics at around 2, 3, 4 and possibly 5 Hz. In an embodiment, an active-pulse is generated at around 12 Hz. As such, a typical venous-induced pulse includes a fundamental around 12 Hz and possible spurious sidebands. Accordingly, a first bandpass filter 510 has a passband 512 so as to generate arterial pulse data 503 at heart rate and heart rate harmonic frequencies 513. Also, a second bandpass filter 510 has a passband 514 so as to generate active-pulse data 504 at the known active-pulse frequency 515.

Also shown in FIG. 5, arterial ratios 520 are calculated from the arterial pulse data 503 so as to generate arterial ratio data 522. In a two wavelength sensor embodiment, arterial ratio data 522 are red/IR ratios. Multiple (more than two) wavelength ratios are described in U.S. Pat. No. 7,343,186 titled Multi-Wavelength Physiological Monitor, assigned to Cercacor and incorporated in its entirety by reference herein. Arterial ratio data 522 are input to an arterial cal curve 530 so as to generate an SpO₂ 532 output. Arterial cal curves are described with respect to FIG. 6A, below.

Further shown in FIG. 5, active-pulse ratios 540 are calculated from the active-pulse data 504 so as to generate active-pulse ratio data 542. In a two wavelength sensor embodiment, active-pulse ratio data 542 are red/IR ratios. Active-pulse ratio data 542 are input to a venous cal curve 550 so as to generate an SpvO₂ 552 output. Venous cal curves are described with respect to FIGS. 6A-B, below.

FIGS. 6A-B illustrate an active-pulse blood analysis system calibration curve (cal curve) 601 embodiment. FIG. 6A illustrates a two-dimensional SpO₂ (arterial) cal curve 610 and a corresponding two-dimensional SpvO₂ (venous) cal curve 620. The SpO₂ cal curve 610 is generated by comparing arterial-pulsed red/IR plethysmograph ratios 602 derived by an optical sensor with corresponding percent oxygen saturation values 603 derived by arterial blood draws analyzed using a calibrated spectrometer. Similarly, the SpvO₂ cal curve 620 is generated by comparing active-pulse red/IR plethysmograph ratios 602 with corresponding percent oxygen saturation values 603 derived by venous blood draws analyzed using the calibrated spectrometer. As examples, a red/IR ratio of 0.6 yields a 96% arterial oxygen saturation value utilizing the arterial cal curve 610, and a red/IR ratio of 0.8 yields a 84% venous oxygen saturation value utilizing the venous cal curve 620.

FIG. 68 illustrates a scatter plot 605 of SpvO₂ 606 versus SvO₂ 607 for an active-pulse optical sensor having greater than two-wavelengths. The scatter plot values 660 compared with a unity line 670 provide a quantitative measure of how well the underlying multi-dimensional cal curve correlates with experimental results.

FIG. 7 illustrates an active-pulse blood analysis system 700 embodiment for advantageously determining SpO₂ and SpvO₂^{A} using the same arterial calibration curve 750. The active-pulse blood analysis system 700 has a sensor data input 701, an SpO₂ output 732 and an SpvO₂^{A} output 752. The bandpass filters 710 generate arterial pulse data 703 and active-pulse data 704 from the sensor data 701, as described with respect to FIG. 5, above. Arterial ratios 720 are calculated from the arterial data 703 so as to generate arterial ratio data 722, and an arterial cal curve 730 is applied to the arterial ratio data 722 so as to generate an SpO₂ 732 output, also described with respect to FIG. 5, above and as described in further detail with respect to FIG. 8A, below.

Further shown in FIG. 7, active-pulse ratios 740 are calculated from the active-pulse data 704 so as to generate active-pulse ratio data 742, as described with respect to FIG. 5, above. Active-pulse ratio data 742 are advantageously input to an arterial cal curve 750 so as to generate an SpvO₂^{A} 752 output, as described in further detail with respect to FIG. 8B, below. Advantageously, the arterial cal curves 730, 750 are the same, as described in further detail with respect to FIGS. 8A-B, below. As described herein, SpvO₂^{A} denotes a venous oxygen saturation measurement utilizing an arterial oxygen saturation cal curve, as set forth with respect to FIG. 4, above.

FIGS. 8A-B illustrate active-pulse blood analysis cal curves for calculating both SpO₂ and SpvO₂^{A}. FIG. 8A illustrates an arterial cal curve for calculating SpO₂. An arterial ratio graph 801 has an arterial ratio x-axis 810, an SpO₂ y-axis 820 and an arterial cal curve 830. The arterial cal curve 830 is numerically-derived by correlating arterial blood draws with corresponding red/IR sensor data responsive to pulsatile arterial blood flow. The cal curve 830 data is derived across a representative patient population and stored in a look-up table. A blood parameter monitor inputs sensor data, derives ratios and calculates corresponding SpO₂ values from the look-up table accordingly. For example, a ratio of 0.75 (812) corresponds to roughly 92% SpO₂ (822); and a ratio of 1.2 (814) corresponds to roughly a 76% SpO₂ (824).

FIG. 88 illustrates an identical arterial cal curve for calculating SpvO₂^{A}. A venous ratio graph 802 has a venous ratio x-axis 840, a SpvO₂^{A} y-axis 850 and the same arterial cal curve 860 stored in a monitor look-up table as described with respect to FIG. 8A, above. However, the arterial cal curve 860 here is used to convert red/IR sensor data measured after attenuation by active-pulse venous blood into derived SpvO₂^{A} values. The rationale for using an arterial cal curve for venous saturation calculations is that the optical characteristics of heart-pulse and active-pulse blood flow are the same. Hence, a ratio of 0.75 (842) corresponds to roughly 92% SpvO₂^{A} (852); and a ratio of 1.2 (844) corresponds to roughly a 76% SpvO₂^{A} (854).

FIG. 9 illustrates an active-pulse blood analysis system 900 embodiment for advantageously determining SpO₂ and SpO₂^{AP} and for combining SpO₂ and SpO₂^{AP} so as to calculate a motion tolerant measure of arterial oxygen saturation. The active-pulse blood analysis system 900 has a sensor data 901 input, an SpO₂ 932 output, an SpO₂^{AP} 952 output, and a motion-tolerant SpO₂^{M} oxygen saturation 972 output. The bandpass filters 910 generate arterial pulse data 903 and active-pulse data 904 from the sensor data 901, as described with respect to FIG. 5, above. Arterial ratios 920 are calculated from the arterial pulse data 903 so as to generate arterial ratio data 922, and an arterial cal curve 930 is applied to the arterial ratio data 922 so as to generate an SpO₂ 932 output, as described with respect to FIG. 5, above.

Further shown in FIG. 9, active-pulse ratios 940 are calculated from the active-pulse data 904 so as to generate active-pulse ratio data 942, as described with respect to FIG. 5, above. Active-pulse ratio data 942 are advantageously input to an active-pulse cal curve 950 so as to generate an SpO₂^{AP} 952 output, as described in further detail with respect to FIGS. 10A-B, below.

Also shown in FIG. 9, a decision logic 960 generates a decision logic output 968. The decision logic output 968 controls a multiplexer 970 that inputs SpO₂ 932 and SpO₂^{AP} 952 so as to generate an SpO₂^{M} output 972 that takes into account both. In an embodiment, a motion indicator 962 and a perfusion indicator 964 are input to the decision logic 960 so that the multiplexer 970 outputs SpO₂^{AP} 952 when a threshold amount of motion 962 and/or perfusion 964 is surpassed and so as to output SpO₂ 932 otherwise. See FIG. 11, below. In this manner, arterial oxygen saturation is advantageously estimated from active-pulse blood flow so as to negate the effect of motion-induced venous blood flow and/or low perfusion. An optical sensor accelerometer for motion detection as well as finger position sensing is described in U.S. patent application Ser. No. 13/646,659 titled Noninvasive Blood Analysis System, cited above.

FIGS. 10A-B illustrates active-pulse blood analysis system cal curve 1001, 1002 embodiments. FIG. 10A illustrates a two-dimensional SpO₂ (arterial) cal curve 1030 and a corresponding two-dimensional SpO₂^{AP} (active-pulse arterial) cal curve 1040. The SpO₂ cal curve 1030 is generated by comparing arterial-pulsed red/IR plethysmograph ratios 1010 derived by an optical sensor with corresponding percent oxygen saturation values 1020 derived by arterial blood draws analyzed using a calibrated spectrometer, as described with respect to FIG. 6A, above. The SpO₂^{AP} cal curve 1040 is generated by comparing active-pulse red/IR plethysmograph ratios 1010 with corresponding percent oxygen saturation values 1020 derived by arterial blood draws analyzed using the calibrated spectrometer. In particular, the SpO₂^{AP} cal curve corresponds relatively well to the SpO₂ cal curve for saturations above about 65%.

FIG. 10B illustrates a scatter plot 1002 comparing noninvasively-derived SpO₂^{AP} values derived with an optical sensor having greater than two-wavelengths with corresponding invasively-derived SaO₂ values. A unity line 1060 provides a measure of quality for the underlying multi-dimensional SpO₂^{AP} cal curve.

FIG. 11 illustrates a motion versus perfusion decision graph 1100 for combining SpO₂ and SpO₂^{AP} so as to calculate a motion and low perfusion tolerant measure of arterial oxygen saturation SpO₂^{M} 972 (FIG. 9). In particular, decision logic 960 (FIG. 9) determines the relative amount of motion 1120 and perfusion 1110 so as to select arterial oxygen saturation SpO₂ 932 (FIG. 9) or active-pulse arterial oxygen saturation SpO₂^{AP} 952 (FIG. 9) as an SpO₂^{M} output 972 (FIG. 9).

As shown in FIG. 11, in a zone embodiment, relative amounts of motion 1120 and perfusion 1110 define discrete zones that determine the use of active pulse. Generally, active pulse (SpO₂^{AP}) 1130 (shaded area) is used as the measure of arterial oxygen saturation (SpO₂^{M}) 972 (FIG. 9) when perfusion is relatively low and/or motion is relatively high. Arterial pulse (SpO₂) 1140 (unshaded area) is used as the measure of arterial oxygen saturation (SpO₂^{M}) 972 (FIG. 9) when perfusion is relatively high and/or motion is relatively low. In a particular zone embodiment, if perfusion 1110 is less than 0.1% 1111, then active pulse 1130 is used regardless of motion 1120. If perfusion 1110 is between 0.1% and 0.5% 1112, then active pulse 1130 is only used if motion is moderate 1122 to severe 1123. If perfusion 1110 is between 0.5% and 10% 1113, then active pulse is only used if motion is severe 1123, and if perfusion 1110 is over 10% 1114, active pulse is not used.

Further shown in FIG. 11, in a boundary embodiment, relative amounts of motion 1120 and perfusion 1110 are specified by a continuous boundary 1150 that determines the use of active pulse. In a particular boundary embodiment, if perfusion 1110 is less than 0.1% 1111, then active pulse 1130 is used regardless of motion 1120, and if perfusion 1110 is over 10% 1114, active pulse is not used. Otherwise, if the combination of increasing motion 1120 and decreasing perfusion 1110 falls below the boundary 1150, then active pulse oxygen saturation 1130 is used as the arterial oxygen saturation SpO₂^{M} output 972 (FIG. 9), and if the combination of decreasing motion 1120 and increasing perfusion 1110 falls above the boundary 1150, then an arterial pulse oxygen saturation 1140 is used as the arterial oxygen saturation SpO₂^{M} output 972 (FIG. 9).

FIG. 12 illustrates another active-pulse blood analysis embodiment for determining SpO₂ and SpO₂^{AP} and for combining SpO₂ and SpO₂^{AP} based upon BSQ (Boolean signal quality) and BPI (Boolean perfusion index) parameters so as to calculate a motion and low perfusion tolerant measure of arterial oxygen saturation SpO₂^{M} (multiplexed oxygen saturation). In particular, FIG. 12 differs from FIG. 9, above, in that the multiplexer (“mux”) select 1303 input is based upon Boolean decision logic 1300 responsive to BWQ 1301 and BPI 1302 inputs.

As shown in FIG. 12, in an embodiment, BSQ=0 indicates low signal quality; BSQ=1 indicates high signal quality; BPI=0 indicates low perfusion; and BPI=1 indicates good perfusion. In an embodiment, BPI=0 when Pl is below 1%. In an embodiment, BSQ is a direct measure of the amount of motion in the signal. In a particular embodiment, accelerometer 350 (FIG. 3) values (x, y and z axis) are compared against a threshold and BSQ=0 when a specified percentage of the samples for any one of the three axis (x, y or z) have an accelerometer output greater than the threshold. In an embodiment, the threshold is 0.3 g and the specified percentage of samples is 50%. Decision logic 1300 is described in detail with respect to FIG. 13, below.

FIG. 13 illustrates a decision logic 1300 embodiment for combining SpO₂ 932 and SpO₂^{AP} 952 inputs into a SpO₂^{M} 972 output. Decision logic 1300 has BSQ 1301 and BPI 1302 inputs as described with respect to FIG. 12, above. SpO₂^{AP} 952 is selected as the SpO₂^{M} 972 output for all combinations of either BSQ=0 or BPI=0, i.e. if either the signal quality or the Pl is low. SpO₂ 932 is selected as the SpO₂^{M} 972 output only if BSQ=1 and BPI=1, i.e. if both the signal quality and the Pl is high.

An active-pulse blood analysis system has been disclosed in detail in connection with various embodiments. These embodiments are disclosed by way of examples only and are not to limit the scope of the claims that follow. One of ordinary skill in art will appreciate many variations and modifications.

1-18. (canceled)

19. A system comprising:
an optical sensor that illuminates a tissue site with multiple wavelengths of optical radiation and that outputs sensor signals responsive to the optical radiation after attenuation by pulsatile blood flow within the tissue site; and
one or more hardware processors configured to analyze the sensor signals to determine an oxygen saturation (SpO2) measurement; and adjust or validate the oxygen saturation measurement based on a signal quality indicator, wherein the signal quality indicator is derived from motion artifacts correlated with perfusion characteristics at the tissue site.

20. The system of claim 19, wherein the optical sensor comprises a photodetector and at least two light sources configured to emit optical radiation at different wavelengths.

21. The system of claim 20, wherein the wavelengths of the optical radiation include at least one red wavelength and one infrared wavelength.

22. The system of claim 19, wherein the hardware processors are further configured to compute the signal quality indicator by analyzing a ratio of motion-induced signal variation to the pulsatile blood flow signal.

23. The system of claim 22, wherein the motion-induced signal variation is determined using accelerometer data integrated into the optical sensor.

24. The system of claim 19, wherein the signal quality indicator is displayed to a user via a graphical or numerical interface.

25. The system of claim 19, wherein the one or more hardware processors are further configured to select a calibration curve for determining the oxygen saturation measurement based on the signal quality indicator.

26. The system of claim 19, wherein the optical sensor is configured for non-invasive placement on a fingertip, earlobe, or forehead.

27. The system of claim 19, wherein the one or more hardware processors apply a rule-based logic to determine the correlation between motion artifacts and perfusion at the tissue site.

28. The system of claim 19, wherein the one or more hardware processors are configured to output an alert when the signal quality indicator falls below a predetermined threshold.

29. The system of claim 19, further comprising a memory storing a library of calibration curves, wherein the hardware processors are configured to dynamically select a calibration curve based on real-time signal characteristics.

30. The system of claim 19, wherein the signal quality indicator is adjusted based on environmental conditions.
